(19) United States
(12) Patent Application Publication
(10) Pub. No.: US 2024/0321410 A1
(43) Pub. Date: Sep. 26, 2024

(54) High-throughput Training Data Generation System for Machine Learning-based Fluid Composition Monitoring Approaches

(71) Applicant: Fluidion US Inc., Los Angeles, CA (US)

(72) Inventor: Dan E. Angelescu, Pasadena, CA (US)

(21) Appl. No.: 18/186,693

(22) Filed: Mar. 20, 2023

(51) Int. Cl. G16C 20/70; G01N 33/18; G06N 20/00

(52) U.S. Cl. CPC G16C 20/70; G01N 33/18; G06N 20/00

(57) ABSTRACT

A system for generating machine learning training data sets for fluid monitoring applications is provided. The system includes two or more containers, each container for storing a fluid sample with a known concentration of one or more additive chemical parameters. A fluidic controller independently controls the flow of the two or more fluid samples, through fluidic conduits, from their respective containers to a mixing unit, in such a way that the relative ratios of the two or more fluid samples delivered to the mixing unit is monitored. A mixer homogenizes the mixture of the two or more fluid samples in the mixing unit to create a homogenously mixed sample. A first sensor performs one or more measurements on the homogenously mixed sample. The system further includes a database and a processor. The processor is configured to: receive as an input, for each fluid sample, the concentration of the one or more additive chemical parameters: instruct the fluidic controller and mixer so as to generate the homogenously mixed sample of the fluid samples; determine a concentration of the one or more additive chemical parameters of the homogenously mixed sample from a combination of the relative ratios of the two or more fluid samples and of the known concentration of their respective additive chemical parameters: obtain results of one or more measurements performed by the first sensor on the homogenously mixed sample; and store in the database the result(s) of the one or more measurements performed by the first sensor, as feature(s), and the concentration of the one or more additive chemical parameter(s) of the homogenously mixed sample and/or the relative ratios of the two or more fluid samples, as label(s).

### TECHNICAL FIELD

The present invention relates to generating training data for machine learning based fluid composition monitoring, and more particularly, to the generation of training data for a neural network for use in fluid composition monitoring.

### BACKGROUND ART

Surface water pollution patterns can be particularly complex, consisting of a wide range of possible chemical and biological contaminants that may be released from either localized or diffuse sources. Surface water pollution sources may be continuous or intermittent; weather and flow patterns (including the operation of various man-made hydraulic structures) may further affect how natural waters and various anthropogenic pollution sources combine at a given location. As a consequence, water samples collected for analysis may contain complex mixtures of contaminants of different types and origins, the relative proportion of which may vary drastically between samples collected at different locations, or at one location but at different times. It is often impossible to determine the relative volumetric proportion in a sample that originates from one or several identified pollution streams.

Surface water quality measurements and associated measurements of pollution patterns or sources, are just one of many examples where fluid mixtures of different origins, and having different chemical compositions, may occur. In such cases, it is important to be able to identify such mixtures, and measure their actual composition. Common examples could occur in drinking water, where finished water from different sources may be blended in a distribution network, or where certain components may be added to the water to change its properties. One specific example is the introduction of potential toxic or poisonous components in the drinking water network, either accidentally or as part of a terrorist attack.

Another example is the adulteration of alcoholic drinks, such as wines, where the perceived taste and quality of a wine can be enhanced artificially through blending of different types of wines, or through adding adulterating compounds or by artificially strengthening its alcohol content. Another example is that of industrial process water, where waters of different chemical compositions or different types of effluents may be mixed together in a single stream, which may need to be characterized. Finally, another example relates to hydrocarbons, such as crude oils, that come in different grades having different market values. In such cases, it is important to be able to identify any attempt to blend different grades of crude, to make it appear as a higher and more expensive grade.

Because composition of a sample may can contain many individual chemical components that could be extremely complex to measure, it is very difficult and costly to measure all the individual components and pollutants that are dissolved in a water sample. Therefore, usually contaminants are classified based on common characteristics, which are characterized through generic techniques. Dissolved organic carbon (DOC) is such a class of chemicals consisting of various organic compounds that may be present in aquatic systems and dissolved in water. Some examples of the many types of organic compounds that can be found in DOC include sugars, proteins, humic substances, alkaloids, phenols etc. The exact composition of DOC can vary depending on the source of the organic matter and the conditions under which it was produced and transported to the aquatic environment. Similarly, nitrates are nitrogen-containing compounds that are often found in water, usually in the form of nitrate ions (NO3−), and provide an essential nutrient for plant growth. Nitrates can enter water sources from various sources such as agricultural runoff, septic systems, livestock operations, industrial discharge. In excess, nitrates can cause eutrophication of water resources which decreases the available dissolved oxygen, harming fish and other aquatic life.

One common technique that is used to quantify DOC, nitrates as well as other classes of chemical contaminants is ultraviolet-visible (UV-visible) spectroscopy, which allows for rapid and quantitative determinations. UV-visible spectroscopy has also been used to measure nitrites, disinfection by-products and as a proxy for estimating chemical and biological oxygen demand of samples. The technique works by shining UV-Visible light through a controlled thickness of a water sample, and measuring the amount of light absorbed by the water sample at different wavelengths. The Beer-Lambert law in Physics correlates the absorbance (defined as the negative base-10 logarithm of the transmittance) at a certain wavelength to the concentrations of the attenuating contaminant species present in the sample and to its thickness. Each contaminant has a different optical absorption spectrum, which can therefore be used to identify and quantify the presence of specific classes contaminants in the water. Optical absorption spectra are generally additive, meaning that the absorbance of a mixture of two components in a given volume of water corresponds to the sum of the absorbances of each individual component separately, as long as no chemical reactions occur between the components. The additivity of light absorption is a fundamental property that is widely used in analytical chemistry in conjunction with the Beer-Lambert law, playing an important role in the analysis of mixtures of chemicals.

However, since the individual UV-visible absorption spectra of different individual components of a class may be similar but are not identical, the absorption spectrum of two mixtures of components from a class, even though they may contain an identical amount of DOC and/or nitrates or other components, may look different. This hinders the ability to accurately measure DOC from the UV-visible absorption spectrum of samples that consist of mixtures from multiple pollution streams.

Different techniques have been developed to deconvolve the UV-visible spectrum and obtain the relative contributions from different classes of components, thus determining the amount of e.g. DOC and nitrates that may be present in a sample (see, for example, Thomas O., Burgess C. 2017 “UV-Visible Spectrophotometry of Water and Wastewater”, Elsevier, which is hereby incorporated herein by reference in its entirety), including techniques based on surrogate measurements performed at a reduced number of wavelengths (see, for example: Carter H T, et al. 2012 “Freshwater DOM quantity and quality from a two-component model of UV absorbance” Water Research, 46(14), pp. 4532-4542; and Causse, J. et al., 2017 “Direct DOC and nitrate determination in water using dual pathlength and second derivative UV spectrophotometry” Water Research, 108(C), pp. 312-319, each of which is hereby incorporated herein by reference in its entirety).

Often, the suspended matter in the sample can strongly interfere with the spectral measurement of the dissolved components, due to light scattering from particles. In this case, sample filtration is required, however filtration for surface waters in field locations is not always trivial, filter fouling being a major issue. Filter fouling can be minimized by using less sample volume per measurement, which can be achieved by using a microfluidic sensor for performing the spectral measurements on the sample. In cases where sample filtration is not possible, different corrections for turbidity need to be applied, which can limit the accuracy of a UV measurement.

Different other types of measurements may be used to determine the chemical composition of a fluid, such as:
Fluorescent spectroscopy based on measuring the emission spectrum, the excitation spectrum, or an excitation-emission matrix.
Spectroscopic measurements in the UV, visible, near-, mid- or far-infrared
Chromatographic measurements, such as HPLC or GC
Arrays of chemical sensors, each with different specificity and selectivity, such as electronic nose or electronic tongue concepts
Various types of electrochemical measurements

The field of chemometrics, defined as the chemical discipline that uses mathematical and statistical methods to design or select optimal measurement procedures and experiments and to provide maximum chemical information by analyzing chemical data (see, for example, Otto, M., 2017. “Chemometrics Statistics and Computer Application in Analytical Chemistry”, Wiley-VCH″ which is hereby incorporated herein by reference in its entirety), has evolved from a need to obtain specific chemical information from data measured with different types of sensors, which are often not fully specific to a component of interest, and which may produce large quantities of data that may not be easy to treat.

Chemometrics is based on the application of mathematical and statistical methods to chemical data analysis. It is a multidisciplinary field that involves chemistry, mathematics, statistics, and computer science, among others. Chemometric techniques are used to analyze chemical data, such as spectra, chromatograms, and chemical images, to extract useful information and insights. Chemometric methods can be used to identify patterns, trends, and relationships in chemical data, to predict properties and behaviors of chemicals, and to optimize chemical processes. Some common chemometric techniques include principal component analysis (PCA), partial least squares regression (PLS), cluster analysis, and discriminant analysis. These techniques can be used in a variety of applications, including analytical chemistry, process control, environmental monitoring, and drug discovery.

Machine learning (ML) has recently emerged as a powerful chemometric technique to model water quality through a variety of supervised and unsupervised learning techniques. See, for example, Guo, Y. et al., 2019 “Advances on Water Quality Detection by UV-Vis Spectroscopy” Applied Sciences, 10(6874), which is hereby incorporated herein by reference in its entirety. ML has been applied to analyzing UV-visible spectra for surface water monitoring. See, for example: Alves, E. M. et al., 2018 “Use of ultraviolet-visible spectrophotometry associated with artificial neural networks as an alternative for determining the water quality index” Environmental Monitoring and Assessment, 190(319), pp. 1-15; and Zhang, H. et al., 2022. “Online water quality monitoring based on UV-Vis spectrometry and artificial neural networks in a river confluence near Sherfield-on-Loddon” Environmental Monitoring and Assessment, 194(630), pp. 1-11., each of which are hereby incorporated herein by reference in its entirety. Approaches have varied from training artificial neural networks (ANN) on features representing direct spectra from a training data set, and using as label the value of the parameter of interest as obtained from a separate laboratory measurement (e.g. DOC, nitrate concentration etc.), to applying principal component analysis (PCA) techniques to reduce the dimensionality of the input parameter space, and using the PCA components to train a ML model. Similarly, partial least squares regression (PLSR) has been applied to model the relationship between the spectra's principal components coefficients and the values of the parameters of interest.

Regardless of the machine learning technique applied, the adequacy of the training data set is essential to guarantee efficient learning and high-quality predictions of parameter values from new spectra. The adequacy of the training data depends on several criteria, some of which are listed below:
1. The quantity of training data provided (number of recorded features or spectra with correct labeling of parameter values)
2. The representativeness of the training data in relation to the variety of potential scenarios that could be encountered
3. The minimization or elimination of spectral artifacts originating from non-absorptive light propagation phenomena (i.e. scattering of light by particles, which would require correction for turbidity)
4. The accuracy of the label assignment (in the case of water quality, that relates to the accuracy of the measurement for different parameters such as DOC, nitrite concentration etc.)
5. The reproducibility of the spectral measurement (i.e. the similarity of the features or spectra obtained from the instrument used in the lab and during field deployment).

In published studies, the training data set relied essentially on natural samples collected at a limited number of field locations over a period of time. However, manual sampling of sufficiently large numbers of surface water samples requires significant logistical and analytical resources, and is unlikely to representatively cover all the possible patterns of contamination, therefore providing only a limited basis for machine learning. Furthermore, due to the difficulty of manual sample collection in severe weather conditions, the resulting training data may be biased towards dry-weather situations, whereas most pollution happens during storm weather events.

The potential contaminant phase space, which depends on the variables that control the particular combination of contaminants at a given location and time, is very large, complex and highly unpredictable. In the case of surface waters, variables that control the water quality at one location and on point in time may include dynamic weather events, local hydrological factors and flow regimes, operation of man-made hydraulic structures, mixing dynamics, operation of the sewage and wastewater treatment infrastructure including combined-sewer overflow, industrial effluent release, agricultural and livestock operations. These represent many potential combinations of pollutant streams, which may appear in an infinity of different relative ratios, thus creating a multi-dimensional contaminant phase space. Some studies have attempted to diversify the available training data from natural samples by supplementing it with samples that were spiked with standards at different concentrations, so as to vary the ranges of different compounds. Such limited sample diversification approaches do not capture the complexity and variability that can be encountered in a natural environment that is prone to pollution from multiple sources, where the water may have different spectral characteristics despite having identical parameter values and, therefore, labels. The current approaches only cover a narrow subset of the total potential contaminant phase space, and are therefore limited in their ability to train models adequately for all possible combinations of pollution sources that may be encountered, and therefore result in predictions that may be inaccurate.

### SUMMARY OF THE EMBODIMENTS

In accordance with an embodiment of the invention, a system for generating machine learning training data sets for fluid monitoring applications is provided. The system includes two or more containers, each container for storing a fluid sample with a known concentration of one or more additive chemical parameters. A fluidic controller independently controls the flow of the two or more fluid samples, through fluidic conduits, from their respective containers to a mixing unit, in such a way that the relative ratios of the two or more fluid samples delivered to the mixing unit is monitored. A mixer homogenizes the mixture of the two or more fluid samples in the mixing unit to create a homogenously mixed sample. A first sensor performs one or more measurements on the homogenously mixed sample. The system further includes a database and a processor. The processor is configured to: receive as an input, for each fluid sample, the concentration of the one or more additive chemical parameters; instruct the fluidic controller and mixer so as to generate the homogenously mixed sample of the fluid samples; determine a concentration of the one or more additive chemical parameters of the homogenously mixed sample from a combination of the relative ratios of the two or more fluid samples and of the known concentration of their respective additive chemical parameters; obtain results of one or more measurements performed by the first sensor on the homogenously mixed sample; and store in the database the result(s) of the one or more measurements performed by the first sensor, as feature(s), and the concentration of the one or more additive chemical parameter(s) of the homogenously mixed sample and/or the relative ratios of the two or more fluid samples, as label(s).

In accordance with related embodiments of the invention, the fluidic controller may be configured to provide fluid sample flow from each container to the mixing unit under the action of gravity, of an external pressure or force applied to the sample, and/or of a vacuum applied to the outlet. The fluidic controller may include individual on-off valves for successively controlling the flow of each fluid sample from the containers to the mixing unit, and further includes one of a scale and a calibrated level meter for measuring the amount of each sample fluid that is being dispensed while the respective valve is in the on position. The fluidic controller may include an individual metering pump and/or an automatic pipetting system, for controlling the individual amounts of the different fluid samples added to the mixing unit. The fluidic controller may be configured to control a flow rate of each individual fluid sample flowing towards the mixing unit. The fluidic controller may include a controllable pump for controlling the flow rate of each fluid sample. The fluidic controller includes one or multiple flow meters installed on the fluid conduits for measuring the flow rate of the fluid samples as they flow towards the mixing unit. The fluidic controller may be configured to have the ability to adjust the individual pressure or force applied to each fluid sample, so as to adjust its flow rate in the respective fluid conduit. The fluidic controller is configured to apply individual pressures to each fluid sample through a pressurizing fluid in gas or liquid phase, said pressurizing fluid being separated from each fluid sample by one of a compliant partition, a piston, and a threaded bag or combinations thereof.

In accordance with further related embodiments of the invention, each fluidic conduit may further include a hydraulic resistor that is selected, based on the respective fluid sample viscosity and the maximum pressure and/or force that the controller can apply to the fluid sample, so as to limit the flow rate of the respective fluid sample to a desired maximum value. The fluidic controller may be configured to control the effective hydraulic resistance of each fluidic conduit, by the means of operating one of a regulating valve, modulating valve, variable constriction, pinch valve, ball valve, globe valve, and needle valve or combinations thereof. The mixer may be an in-line mixer configured to homogenize the different fluid sample streams on the fly, wherein the first sensor is an in-line sensor configured to measure the resulting homogenously mixed sample while it flows, and wherein the processor is configured to determine the relative ratios of the two or more fluid samples present in the homogenously mixed sample stream from their respective instantaneous flow rates. At least one of the fluid conduits, mixing unit, mixer and first sensor is a microfluidic device with channels having lateral dimension between 1 μm and 1000 μm. The processor may automatically generate a selection of fluid sample combinations that are directed to a predetermined portion of the phase space of possible combinations. The predetermined portion of the phase space targets ranges of samples combinations based on their likelihood of being encountered in real-life situations. The database may be located on a remote computer server which communicates with the processor via a wireless or wired communication link.

In accordance with yet further related embodiments of the invention, the system may include a machine learning system trained on the data stored in the database, for predicting at least one of the one or more chemical parameters values and the relative ratios of two or more fluid samples, from measurements performed on new fluid samples. A copy of the machine learning system may be trained on the data, for remote use with a second sensor similar to the first sensor. The fluid samples may be acquired in an operational environment and do not have a known concentration of one or more additive chemical parameters. The machine learning system may be located on a remote computer server, the first sensor transmitting the measurements to the server via a wireless or wired communication link, and the server transmitting back the one or more chemical parameters values predicted by the machine learning system. The machine learning system may be at least partially based on an artificial neural network. The machine learning system may implement partial least squares regression.

In still further related embodiments of the invention, the processor may be further configured to determine an error estimate based on comparing the predicted and the determined parameter values for one or more new sample fluids with a known concentration of one or more additive chemical parameters, but that are not yet included in the database. The processor may be further configured to select new fluid sample combinations to generate new features and labels to include in an enlarged training database; re-train the machine learning system on the enlarged training database; recalculate a new error estimate, and iteratively repeat the process of selecting, re-training and recalculating until the error estimate decreases below a desired precision threshold, or a maximum number of iterations is reached.

In further related embodiments of the invention, the two or more fluid samples are selected from one of: surface water matrices likely to be encountered at a specific location and/or pollutant streams known to contaminate the respective surface water matrices, hydrocarbon mixtures found in downhole oilfield exploration, refining operations and/or produced crude oils, an alcoholic beverage combined with a product used to adulterate alcoholic beverages, pure drinking water combined with pollutants, contaminants, chemical warfare agents, nerve agents, toxic and/or poisonous compounds, and pure air combined with pollutants, chemical warfare agents, nerve agents, toxic and/or poisonous compounds. The first sensor may be one of: an optical spectrometer, and the measurement includes an absorbance spectrum measured in the ultraviolet, visible, near infrared, mid infrared and/or far infrared spectral ranges; a fluorescence spectrometer, and the measurement includes an emission spectrum, an excitation spectrum, or an excitation-emission matrix; a Ramon spectrometer; a chromatography system, and the measurement includes a chromatograph recorded with a detector; and a chemical sensor array. The system may include a cleaning mechanism for cleaning and/or decontaminating the mixing unit, mixer and/or first sensor between successive measurements.

In accordance with another embodiment of the invention, a method of generating machine learning training data sets for fluid monitoring applications is provided. The method includes mixing two or more fluid samples to generate a homogenously mixed sample such that the relative ratios of the two or more fluid samples that are mixed is known, each fluid sample having a known concentration of one or more additive chemical parameters. A concentration of the one or more additive chemical parameters of the homogenously mixed sample is determined from a combination of the relative ratios of the two or more fluid samples and of the known concentration of their respective additive chemical parameters. Results of one or more sensor measurements are obtained on the homogenously mixed sample. The method further includes storing in a database the result(s) of the one or more measurements, as feature(s), and the concentration of the one or more additive chemical parameter(s) of the homogenously mixed sample and/or the relative ratios of the two or more fluid samples, as label(s).

In accordance with relate embodiments of the invention, each fluid sample may be stored in a separate container, wherein mixing two or more fluid samples includes providing flow of fluid sample from each container to a mixing unit under the action of gravity, of an external pressure or force applied to the sample, and/or of a vacuum or suction pump applied to the outlet. Providing flow of fluid sample from each container to the mixing unit may include controlling the flow of fluid sample with individual on-off valves, and may further include measuring the amount of each sample fluid that is being dispensed while the respective valve is in the on position. Providing fluid sample flow from each container to the mixing unit may include using an individual metering pump and/or an automatic pipetting system, for controlling the individual amounts of the different fluid samples added to the mixing unit. Providing fluid sample flow from each container to the mixing unit may include controlling a flow rate of each individual fluid sample flowing towards the mixing unit. Controlling the flow rate of each individual fluid sample flowing towards the mixing unit may include using a controllable pump for controlling the flow rate of each fluid sample. Controlling the flow rate of each individual fluid sample flowing towards the mixing unit may include measuring the flow rate of the fluid samples as they flow towards the mixing unit. Controlling the flow rate of each individual fluid sample flowing towards the mixing unit may include adjusting the individual pressure or force applied to each fluid sample. Adjusting the individual pressure or force applied to each fluid sample may include applying individual pressures to each fluid sample through a pressurizing fluid in gas or liquid phase, said pressurizing fluid being separated from each fluid sample by one of a compliant partition, a piston, and a threaded bag or combinations thereof. Controlling the flow rate of each individual fluid sample flowing towards the mixing unit may include using a hydraulic resistor. Controlling the flow rate of each individual fluid sample flowing towards the mixing unit may include controlling the effective hydraulic resistance using one of a regulating valve, a modulating valve, a variable constriction, a pinch valve, a ball valve, a globe valve, and a needle valve or combinations thereof.

In accordance with further related embodiments of the invention, mixing two or more fluid samples to generate a homogenously mixed sample may includes homogenizing different fluid sample streams on the fly, wherein obtaining results of one or more sensor measurements on the homogenously mixed sample includes measuring the resulting homogenously mixed sample while it flows, and wherein the relative ratios of the two or more fluid samples present in the homogenously mixed sample stream is determined from their respective instantaneous flow rates. The method may include using microfluidic device with channels having lateral dimension between 1 μm and 1000 μm. A selection of fluid sample combinations for mixing may be generated that are directed to a predetermined portion of the phase space of possible combinations. The predetermined portion of the phase space may target ranges of samples combinations based on their likelihood of being encountered in real-life situations. A processor may control and/or perform the mixing, determining, obtaining and storing, and the database may be located on a remote computer server which communicates with the processor via a wireless or wired communication link.

In accordance with still further related embodiments of the invention, the method may further include training a machine learning system on the data stored in the database. The machine learning system may predict the one or more chemical parameters values and/or the relative ratios of two or more fluid samples, from measurements performed on new fluid samples not included in the training. A copy of the machine learning system may be trained on the data, for remote use. The new fluid samples may be acquired in an operational environment. The machine learning system may be located on a remote computer server, the method further including sending the results of the one or more sensor measurements to the server via a wireless or wired communication link, and transmitting back, by the server, the one or more chemical parameters values predicted by the machine learning system. The machine learning system may be at least partially based on an artificial neural network. The machine learning system may implement partial least squares regression.

In accordance with yet further related embodiments of the invention, the method may include determining an error estimate based on comparing the predicted and the determined parameter values for new fluid sample combinations that are not yet included in the database, which have a known concentration of one or more additive chemical parameters. The method may further include: selecting new fluid sample combinations to generate new features and labels to include in an enlarged training database; re-training the machine learning system on the enlarged training database; recalculate a new error estimate, and iteratively repeat the process of selecting, re-training and recalculating until the error estimate decreases below a desired precision threshold, or a maximum number of iterations is reached.

In further related embodiments of the invention, the two or more fluid samples are selected from one of: surface water matrices likely to be encountered at a specific location and/or pollutant streams known to contaminate the respective surface water matrices, hydrocarbon mixtures found in downhole oilfield exploration, refining operations and/or produced crude oils, an alcoholic beverage combined with a product used to adulterate alcoholic beverages, pure drinking water combined with pollutants, contaminants, chemical warfare agents, nerve agents, toxic and/or poisonous compounds, and pure air combined with pollutants, chemical warfare agents, nerve agents, toxic and/or poisonous compounds. Obtaining results of one or more sensor measurements on the homogenously mixed sample may include using one of: an optical spectrometer, and the measurement includes an absorbance spectrum measured in the ultraviolet, visible, near infrared, mid infrared and/or far infrared spectral ranges; a fluorescence spectrometer, and the measurement includes an emission spectrum, an excitation spectrum, or an excitation-emission matrix; a Raman spectrometer; a chromatography system, and the measurement includes a chromatograph recorded with a detector; and a chemical sensor array. The method may be performed by a system, the method further including cleaning and/or decontaminating the system between successive measurements.

In accordance with another related embodiment of the invention, the method may be performed by the above-described system.

### BRIEF DESCRIPTION OF THE DRAWINGS

The foregoing features of embodiments will be more readily understood by reference to the following detailed description, taken with reference to the accompanying drawings, in which:

FIG. 1 shows a system for automatically generating dense machine learning training data sets for fluid monitoring applications, in accordance with an embodiment of the invention;

FIG. 2 shows a fluidic controller, in accordance with an embodiment of the invention;

FIG. 3 shows a system for automatically generating dense machine learning training data sets for fluid monitoring applications, where the mixer and/or the first sensor 303 is installed in-line, in accordance with an embodiment of the invention;

FIG. 4 shows a fluidic controller that includes multiple flow meters for measuring the flow rates of the respective fluid samples as they flows towards the mixer, in accordance with an embodiment of the invention;

FIG. 5 shows various configurations of sample containers and associated fluidic controller that allow for pressure control of individual samples, in accordance with an embodiment of the invention.

FIG. 6 shows a fluidic controller configured to control the effective hydraulic resistance of the fluidic conduits, in accordance with an embodiment of the invention;

FIG. 7 displays a system for automatically generating dense machine learning training data sets for fluid monitoring applications that further includes a machine learning system that has been trained on data stored in the database, in accordance with an embodiment of the invention;

FIG. 8 is a flow diagram of a methodology for training the machine learning system, in accordance with an embodiment of the invention;

FIG. 9 shows a system for automatically generating dense machine learning training data sets for fluid monitoring applications wherein the database and a machine learning system are located on a remote server, in accordance with an embodiment of the invention;

FIG. 10 shows a system that includes a copy of the machine learning system 1002 trained on the data, that is configured for use with a second sensor, in accordance with an embodiment of the invention; and

FIG. 11 depicts an exemplary protocol for using the second sensor in the field to analyze an unknown sample, in accordance with an embodiment of the invention.

### DETAILED DESCRIPTION OF SPECIFIC EMBODIMENTS

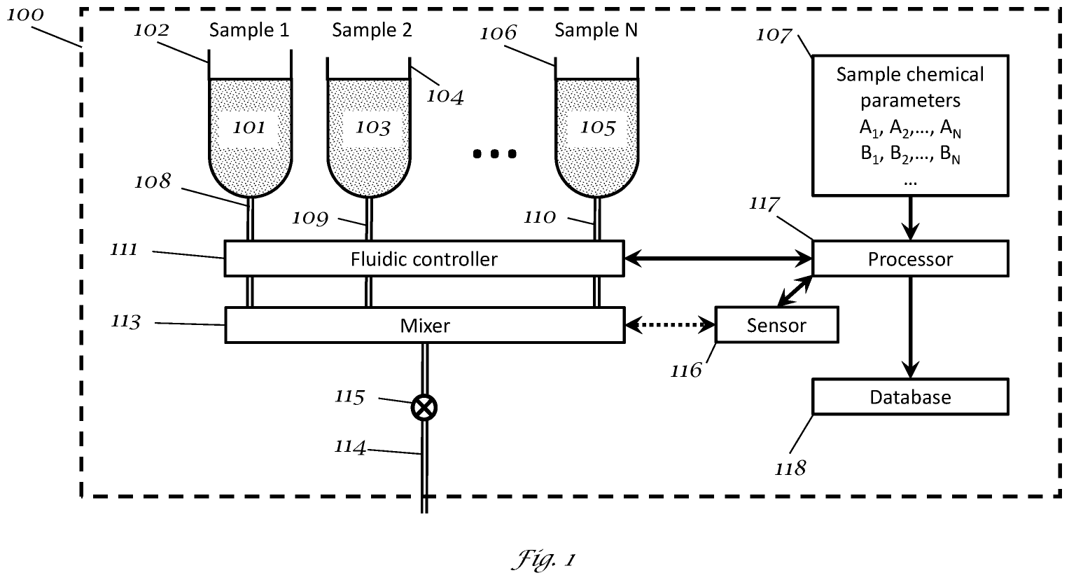

FIG. 1 shows a system 100 for automatically generating dense machine learning training data sets for fluid monitoring applications, in accordance with an embodiment of the invention. The system is designed to combine two or more fluid samples 101, 103, . . . , 105, labeled Sample 1, Sample 2, . . . , Sample N (N≥2), each having a known concentration of one or several chemical parameters, or constituents: A₁, A₂ . . . . A_{N}; B₁, B₂ . . . . B_{N}; etc. that are provided as an input 107 to the system and accessible to a processor 117. The fluid samples 101, 103 . . . 105 may be in liquid phase or in gas phase at ambient pressure and temperature conditions.

The chemical parameters described above are presumed to be additive, meaning that no chemical reaction is expected to occur between the different chemical constituents upon mixing of the samples, so that the total quantity of a specific constituent in a mixture of samples is equal to the sum of the quantities of that constituent present in each of the samples mixed. In mathematical terms, the additive property implies that, if volumes V₁, V₂ . . . . V_{N} of Sample 1, Sample 2, . . . , Sample N are mixed together, and the samples are assumed to be incompressible, then the resulting sample will have a volume V=V₁+V₂+ . . . +V_{N} and the resulting concentration of the chemical parameters in the resulting sample will be given by A=(A₁×V₁+A₂×V₂+ . . . +A_{N}×V_{N})/V; B=(B₁×V₁+B₂×V₂+ . . . +B_{N}×V_{N})/V; etc. In other words, if the relative ratios Vf₁, Vf₂ . . . Vf_{N} of Sample 1, Sample 2, . . . . Sample N, calculated as Vfᵢ=Vᵢ/V (i=1, 2 . . . . N), are known, then the concentration of the mixture can be calculated by the weighted sum of their concentrations: A=A₁×Vf₁+A₂×Vf₂+ . . . +A_{N}×Vf_{N}; B=B₁×Vf₁+B₂×Vf₂+ . . . +B_{N}×Vf_{N} etc. It results that knowledge of the concentrations of an additive chemical parameter in each of the samples to be mixed, and of their relative ratios in the final mixture, allows for determination of the concentration of the respective chemical parameter of the final mixture. These relationships hold identically if the concentrations are replaced by mass fractions, and the volumes of the samples and mixture by their respective masses. The known densities of the samples D₁, D₂ . . . D_{N} can be used to relate the volume Vᵢ of each sample to its mass Mᵢ: Dᵢ=Mᵢ/Vᵢ, and may also be provided as part of the input 107 to the system 100. Such conversions are well known to the person skilled in the art, who will recognize that the embodiments described herein can be trivially adapted to using concentrations, mass ratios, molar concentration or other notions of concentration as may be customary in a specific field. Similarly, if the flow rates F₁, F₂, . . . , F_{N} of fluid samples are known, and the fluid samples are combined and homogenized, then the resulting flow rate of homogenized mixture can be calculated as F=F₁+F₂+ . . . +F_{N} and the concentrations of the different additive chemical parameters can be inferred as A=(A₁×F₁+A₂×F₂+ . . . +A_{N}×F_{N})/F; B=(B₁×F₁+B₂×F₂+ . . . +B_{N}×F_{N})/F; etc.

The fluid samples 101, 103, . . . , 105 are stored in separate containers 102, 104, . . . , 106, which are in turn connected through separate fluidic conduits 108, 109, . . . , 110 to a fluidic controller 111, and further to a mixing unit including a mixer 113. The operation of the fluidic controller 111 and optionally that of the mixer 113 is coordinated by the processor 117, the fluidic controller 111 being, without limitation, configured to independently control the flow of the two or more fluid samples from their respective containers to a mixing unit, in such a way that the relative ratios of the two or more fluid samples is monitored by the processor 117, and the mixer 113 being configured to mix the samples exiting the fluidic controller and provide a homogenously mixed sample. A first sensor 116 is configured to perform one or more measurements on the homogenously mixed sample, and transmit the results back to processor 117. At the end of the measurements, the homogenously mixed sample is ejected from the mixer and the system 100 through outlet 114, which may optionally be configured to integrate an outlet valve 115 controlled by the processor 117.

The system 100 may further include a mechanism for cleaning and/or decontaminating the mixing unit, mixer and/or first sensor between successive measurements, so as to avoid cross-contamination between successive samples. Such cleaning mechanism may involve a cleaning procedure consisting of one or multiple steps selected from: a flushing step using one of a clean fluid and a solvent, a drying step, a mechanical brushing step, an ultrasonic cleaning step, a UV disinfection step, a chemical disinfection step, a heating step. The cleaning procedure may involve any other cleaning steps known in the art.

The processor 117 is further in communication with a database 118 to store the result(s) of the one or more measurements performed by the first sensor, as feature(s), and the concentration of the one or more additive chemical parameter(s) of the homogenously mixed sample as label(s). The relative ratio(s) of the two or more fluid samples mays also be stored as label(s) in the database 118. Database 118 may be local to the system 100, or, in a related embodiment of the invention, it may installed on a remote computer server which communicates with the processor via a wireless or wired communication link.

In various embodiments, the first sensor 116 may be an optical spectrometer for a specific range of the light spectrum and is paired with an appropriate broadband, visible, infrared (IR) or programmable light source or laser source, and the processor may be configured to calculate the absorbance spectrum from the measured spectrum of the homogenously mixed sample and a blank spectrum measured on a clean sample such as a deionized ultrapure water sample. The system 100 may be further configured so that one of the fluid samples 101, 103 . . . 105 consists of the clean sample, and the blank is measured by the first sensor periodically during its operation. In related embodiments, the first sensor 116 may be one of a UV spectrometer, a visible spectrometer, a near-IR spectrometer, a mid-IR spectrometer, a far-IR spectrometer, a Fourier-transform IR (FTIR) spectrometer, or a Raman spectrometer, or a combination thereof. In other embodiments, the first sensor 116 may include a fluorescence spectrometer configured to measure the emission spectrum of the sample, the excitation spectrum, or an excitation-emission matrix. First sensor 116 may also use other types of spectroscopy systems known to the person skilled in the art, or may integrate several different types of optical spectrometers into a hybrid sensor.

In various embodiments, the first sensor 116 may be a chromatography system combined with an appropriate detector, configured to measure a sample chromatogram. The chromatography system may be one of a high-performance liquid chromatography (HPLC) system and a gas chromatography (GC) system, outfitted with an separation column adapted to the chemical nature of the samples and chemical parameters of interest, and optionally configured to sample from the headspace of the homogenously mixed sample. The detector may include one of a UV-visible detector, a mass spectrometer (MS), a flame ionization detector (FID), a photodiode array, a fluorescence sensor, a refractive index, electrochemical and an electrical conductivity probe. Other types of chromatography systems, sampling techniques and detectors known to the person skilled in the art may be combined.

The first sensor 116 may include a chemical sensor array, each element of the array sensitive to different chemical properties of a sample or to different chemical compounds present in the sample, and having different degrees of sensitivity and specificity to different chemical compounds. Such chemical sensor array may be configured as an electronic nose, also known as an e-nose, and include elements that detect and analyze the various volatile organic compounds (VOCs) present in the homogenously mixed sample (if it is in gas phase) or in the associated headspace. Alternatively, if the homogenously mixed sample is in liquid phase, the chemical sensor array may be configured as an electronic tongue to detect different chemical properties of the sample, specifically those that are related to pollutants and toxins present in the sample.

Multiple sensors using different technologies and measurement principles, including, but not limited to the ones listed in the embodiments presented above, may be combined, resulting into a hybrid first sensor 116.

The mixer 113 may be an active mixer, requiring an external energy source, which may integrate: an agitator, a homogenizer, a magnetic stirrer, an ultrasonic generator, a blender, and/or an impeller, or another active mixing device known in the art.

Alternatively, the mixer 113 may be a passive mixer, requiring no external energy, which may integrate: a helical mixer, a baffle mixer, a junction mixer, a split-and-recombine mixer optionally employing flow-folding principles, a pinch channel mixer, a combination thereof, or any another passive mixing device known in the art. The passive mixer may be an in-line mixer.

At least one of the fluid conduits 108, 109 . . . 110, mixing unit 204, mixer 113 and first sensor 116 may be a microfluidic device with channels having lateral dimension between 1 μm and 1000 μm. The person skilled in the art will recognize that the miniaturization of the fluidic and sensor part of the system 100 using microfluidic technology will reduce the amount of fluid sample required for each sample measurement, therefore increasing the amount of data that can be generated from a given set of fluid sample amounts that are provided to the system. The person skilled in the art will further recognize that a microfluidic geometry can be advantageously used in conjunction with filters on the fluid conduits, and/or at the inlet of the sensor, so as to eliminate suspended matter that may interfere with an optical absorption measurement. The smaller fluid sample volumes required with a microfluidic system will in this case allow a larger number of measurements to be performed prior to filter fouling.

The fluidic controller 111 may include an individual metering pump and/or an automatic pipetting system, for controlling the individual amounts of the different fluid samples added to the mixing unit. Alternatively, another device known in the art for dispensing precise amounts of fluid may be used. The fluidic controller 111 may be configured to control a flow rate of each individual fluid sample flowing towards the mixing unit.

In various embodiments, fluid samples 101, 103 . . . 105 may include surface water matrices likely to be encountered at a specific location and/or pollutant streams known to contaminate the respective surface water matrices. The fluid samples may be hydrocarbon mixtures found in downhole oilfield exploration, refining operations and/or produced crude oils. The fluid samples may include alcoholic beverages, including their combinations with certain products used to adulterate such beverages. The fluid samples may include drinking water and water combined with pollutants, contaminants, chemical warfare agents, nerve agents, toxic and/or poisonous compounds. In further embodiments, the fluid samples may be in a gas phase and include pure air and air combined with pollutants, chemical warfare agents, nerve agents, toxic and/or poisonous compounds.

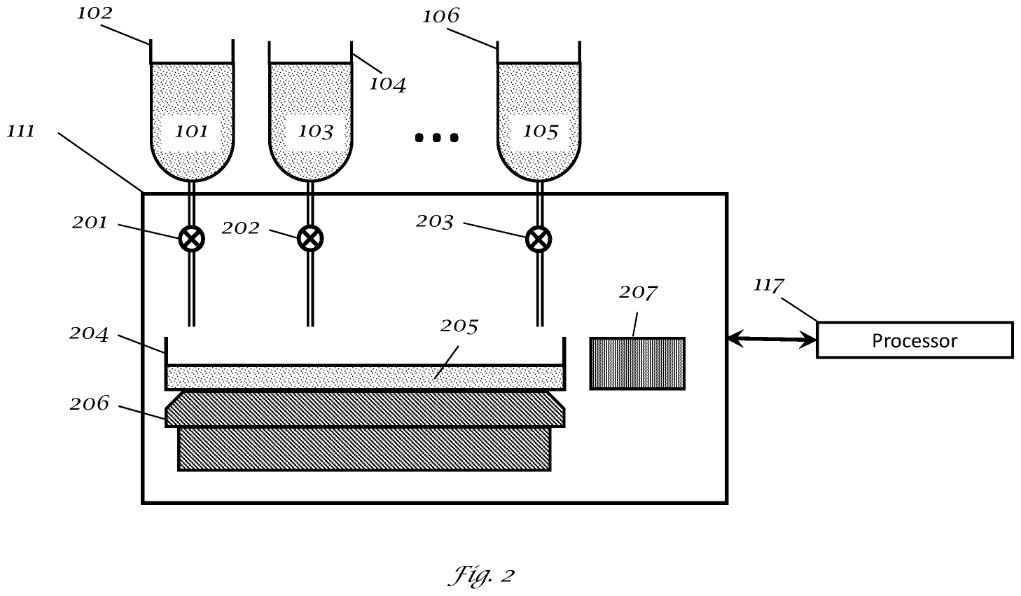

FIG. 2 shows an embodiment of the fluidic controller 111, including individual on-off valves 201, 202 . . . 203 for controlling the flow of the fluid samples 101, 103 . . . 105 from the containers 102, 104, . . . , 106 to the mixing unit 204. The on-off valves corresponding to each fluid sample are configured to turn on successively for a pre-defined amount of time, and dispense an amount of the respective sample into the mixing unit. The controller further includes one of a scale 206 and a calibrated level meter 207 for measuring the amount of each sample fluid that is being dispensed while the respective valve is in the on position, and further transmits the measured amounts to the processor 117 which uses them to calculate the relative ratios of the sample fluids that will compose the homogenously mixed sample 205.

Samples 101, 103, . . . , 105 may be driven by gravity flow, the containers 102, 104 . . . 106 being placed at heights relative to the fluidic controller that provide sufficient hydrostatic pressure for ensuring adequate operation. In various embodiments, the processor and/or fluidic controller may include the ability to control the heights of the different containers so as to control their respective hydrostatic pressure and the resulting flow rates while valves 201, 202 . . . 203 are turned on.

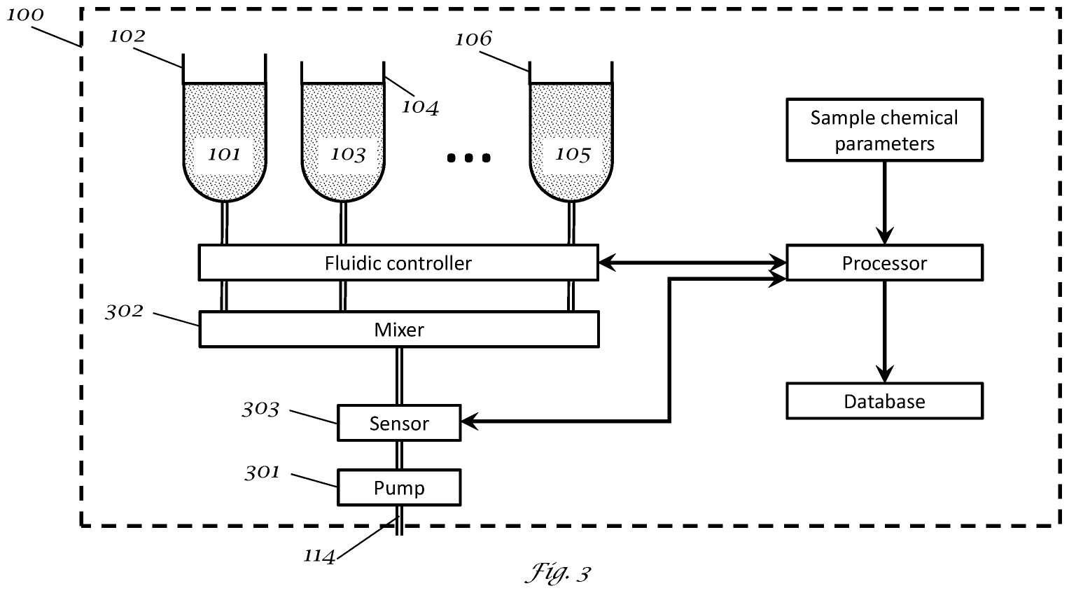

FIG. 3 shows another embodiment of the system 100, where at least one of the mixer 302 and the first sensor 303 are installed in-line, the mixer 302 configured to mix the fluid samples provided by the fluidic controller and produce the resulting homogenously mixed sample on-the-fly, and the first sensor configured to measure the homogenously mixed sample as if flows through.

The system 100 may be further equipped with a vacuum reservoir or a suction pump 301 near the outlet 114, such that the respective vacuum or suction pressure provides the driving force for driving the fluid samples through the system.

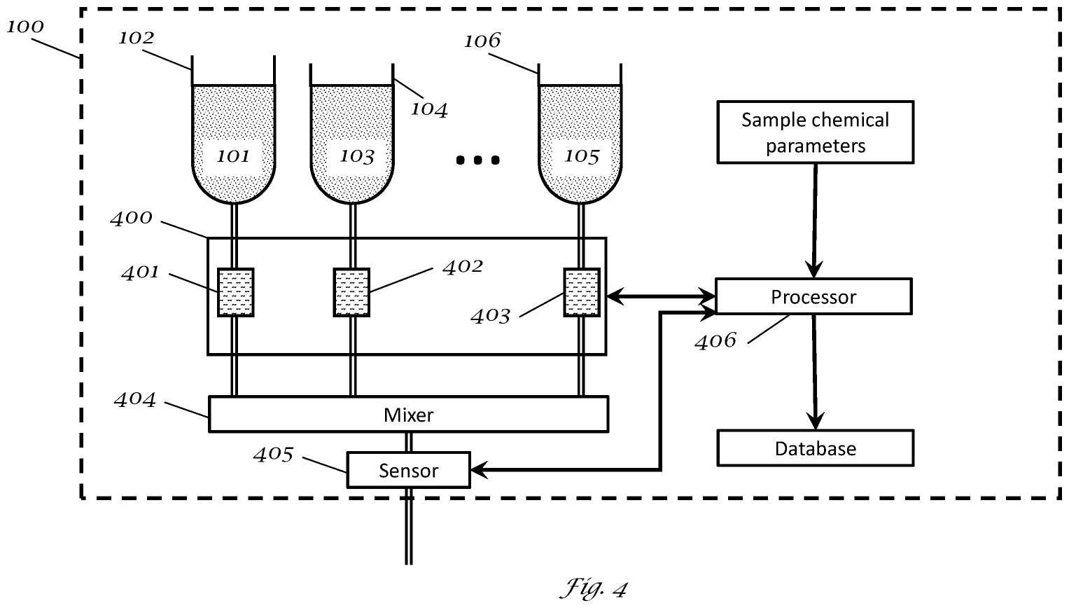

FIG. 4 displays another possible embodiment of the fluidic controller 400, wherein the fluidic controller 400, mixer 404 and first sensor 405 are installed in-line, the mixing unit and mixer 404 configured to mix the fluid samples provided by the fluidic controller 400 and produce the resulting homogenously mixed sample on-the-fly, the first sensor 405 configured to measure the homogenously mixed sample as it flows through, and the fluidic controller 400 further including, on the fluidic conduits, multiple flow meters 401, 402 . . . 403 for measuring the flow rates of the respective fluid samples 101, 103, . . . , 105 as they flows towards the mixer. The processor 406 then calculates the relative ratios of the fluid samples and the concentration of the one or more additive chemical parameters on-the-fly, from the instantaneous flow rates of each fluid sample as provided by their respective flow meters 401, 402 . . . 403.

The fluidic controller may include, on each fluidic conduit, a controllable pump for controlling the flow rate of each fluid sample in real time. The processor may then calculate the relative ratios of the fluid samples and the concentration of the one or more additive chemical parameters from the instantaneous flow rates of each fluid sample as controlled by the fluidic controller.

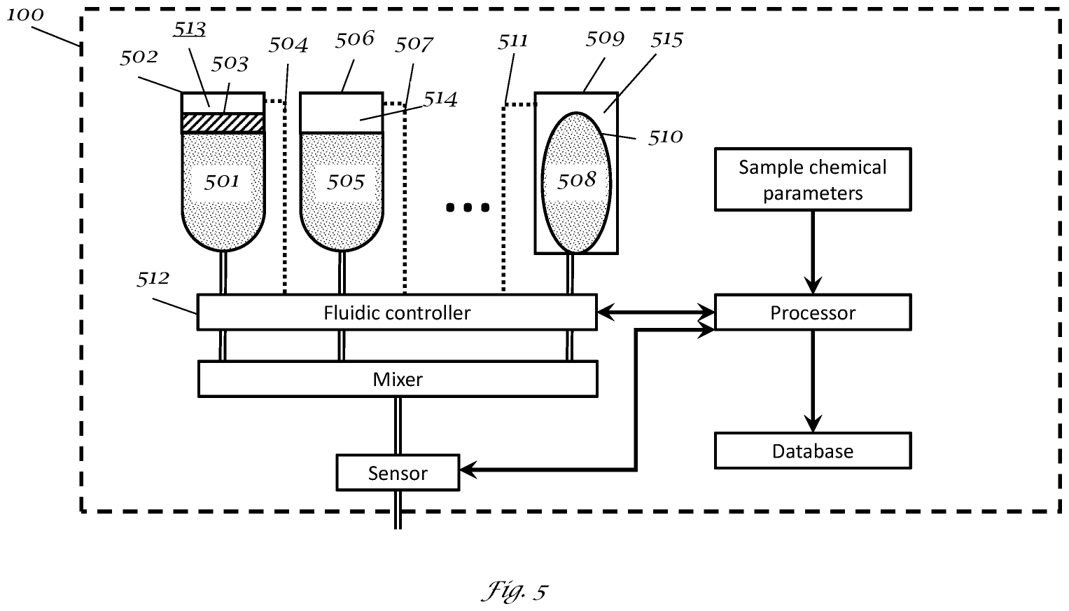

In various embodiments, the system 100 may further have the ability to apply different pressures or forces to each fluid sample, so as to control their individual flow rates. FIG. 5 shows several possible exemplary configurations of the sample containers 502, 506 . . . 509 and of the fluidic controller 512 for allowing pressure control of the individual samples 501, 505 . . . 508.

The sample container 502 may be a closed container with the fluidic controller 512 having the means to pressurize the container 502 using a separate pressurizing fluid 513 supplied through a separate conduit 504. The pressurization fluid 513 may be a liquid or a gas. The said pressurization means may consist of an air pump and means to measure and control its output pressure, a controllable regulator connected to an external pressurized gas line, a pressure controller, a syringe pump controller, or any other similar pressurization device known in the art. The container 502 may further include a movable sealed piston 503, which physically separates the pressurizing fluid 513 from the sample fluid 501, while transmitting its pressure.

In various embodiments, the closed container 506 may be filled with a fluid sample 505, the pressurization fluid 514 supplied by fluidic controller 512 through conduit 507 being immiscible with the fluid sample 505 and having a direct contact interface with it. In related embodiments, fluid sample 505 is a liquid sample and the pressurization fluid 514 is a gas chosen with low solubility in the liquid sample.

In a further related embodiment, the closed container 509 may include a compliant partition 510 between a first volume filled with fluid sample 508 and a second volume filled with pressurization fluid 515 that is supplied by fluidic controller 512 through conduit 511, the compliant partition physically separating the pressurizing fluid from the sample fluid while transmitting its pressure. The compliant partition may be designed so as to easily deform under an applied pressure difference, such as in the form of a thin unstressed foil or plastic sheet. In further embodiments, the compliant partition is part of a threaded bag.

The fluidic controller may include fixed, known, hydraulic resistances on the different fluidic conduits to control the flow rates of the respective fluid samples under the effect of the pressure provided by the pressurization fluid. Such hydraulic resistors may be selected so as to limit the flow rate of each fluid sample to a desired maximum value, corresponding to the maximum pressure that can be applied via the pressurization fluid. In illustrative embodiments, such hydraulic resistors include thin capillaries or microchannels in a microfluidic chip, of sufficiently small effective diameter as to ensure laminar flow of the fluid sample through the hydraulic resistor.

The person skilled in the art will recognize that a large variety of combinations of the above embodiments, as well as other systems or devices known in the art, could be used to provide the same function of separating the pressurization fluid from the sample fluid while transmitting its pressure, and that the described embodiments are exemplary.

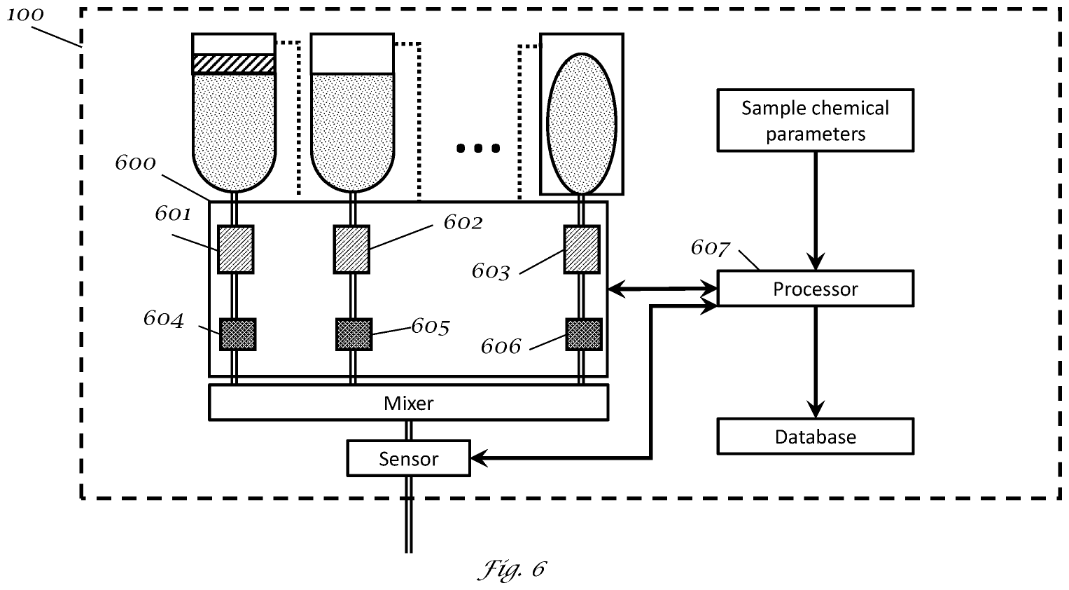

FIG. 6 shows a fluidic controller 600 configured to control the effective hydraulic resistance of the fluidic conduits, using various operating variable constrictions 601, 602 . . . 603 on the fluidic conduits, which allows control of their respective flow rates. Such variable constriction may include a regulating valve, modulating valve, pinch valve, ball valve, globe valve, needle valve, or combinations thereof, as well as any other device known in the art to achieve a similar function.

The fluidic controller 600 may further include, on the fluidic conduits, multiple flow meters 604, 605 . . . 606 for measuring the instantaneous flow rates of the respective fluid samples, processor 607 being configured to calculate the relative ratios of the fluid samples and the concentration of the one or more additive chemical parameters on-the-fly, from the instantaneous flow rates of each fluid sample as provided by their respective flow meters.

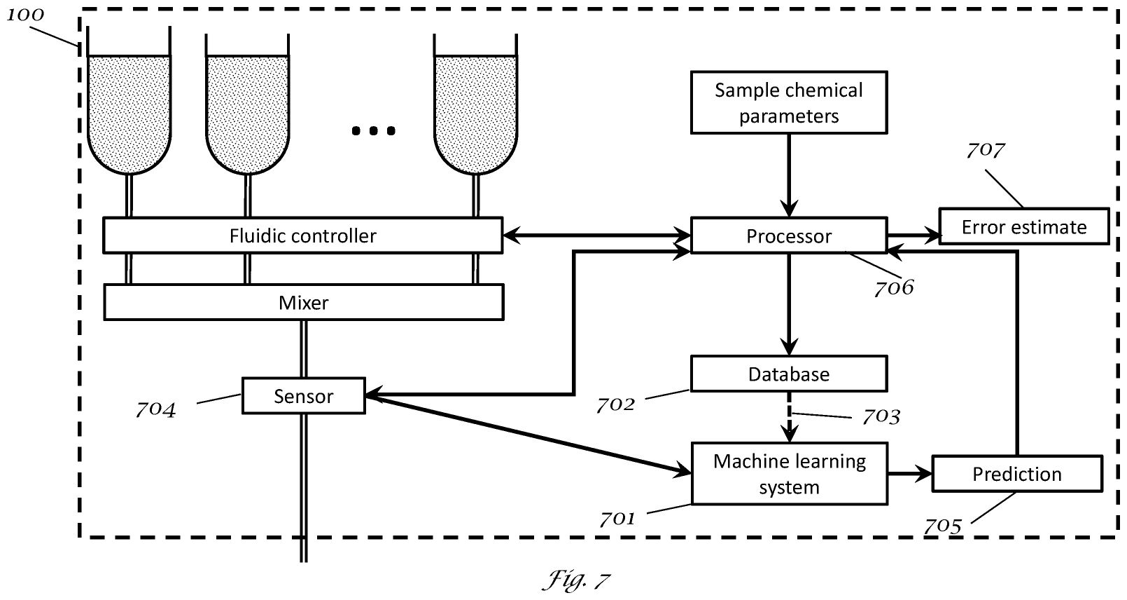

FIG. 7 displays another embodiment of the invention, including a machine learning system 701 that has been trained 703 on data stored in the database 702. Illustratively, the machine learning system 701 may be able to make predictions 705 of chemical parameter values from measurements performed by the first sensor 704 on new fluid samples produced by the system 100, but not included in the training data.

The machine learning system 701 may be an artificial neural network that is trained on the data from the database using the sensor measurements performed on samples, as features, and using the concentration of the one or more additive chemical parameter(s) of the samples and/or the relative ratios of the corresponding fluid samples as label(s). In a further related embodiments, the machine learning system 701 may implement a partial least squares regression algorithm.

The processor 706 may be configured to automatically determine an error estimate 707 based on comparing the predicted parameter values 705 and the determined parameter values for one or more new homogenously mixed sample produced by the system, but that were not included in the training data for the machine learning system 701. In various embodiments, the error estimate 707 may be determined as the root mean square error.

The system 701 may be configured to automatically train itself to reach a desired level of accuracy. To this end, the processor 706 may be configured to automatically select new fluid sample combinations to measure and generate new features and labels to include in an enlarged training database, re-train the machine learning system 701 on the enlarged training database, recalculate a new error estimate 707 and iteratively repeat the process of selecting, re-training and recalculating until the error estimate 707 decreases below a target error threshold (which indicates that the desired level of accuracy has been reached), or a maximum number of iterations Nₘₐₓ is reached.

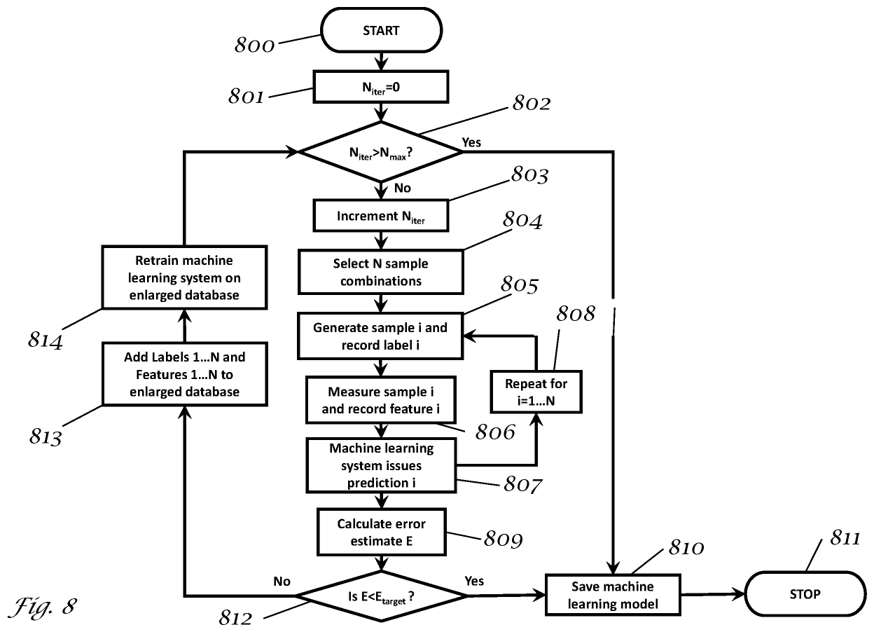

FIG. 8 is an exemplary flow diagram representing this training process, which may be automatic, in accordance with an embodiment of the invention. A target error threshold E_{target} along with a maximum number of iterations Nₘₐₓ and a number of samples in a learning batch, N, are established upon starting the automatic training process. An initial version of a trained machine learning system is also provided. Upon starting the automatic training process (step 800), the processor sets the iterations counter Nᵢₜₑᵣ to 0 (step 801). Then, it compares (step 802) the current iteration number Nᵢₜₑᵣ to the maximum number of iterations Nₘₐₓ and, if Nᵢₜₑᵣ>Nₘₐₓ, it saves the current version of the machine learning model (step 810) and stops (step 811) without achieving the training goal. If the condition Nᵢₜₑᵣ>Nₘₐₓ is not satisfied at step 802, then a new iteration is started by incrementing the current iteration number Nᵢₜₑᵣ (step 803) and selecting a new batch of N sample combinations (step 804). Then for each new fluid sample combination Sample i in the new batch, the fluidic controller and mixer are instructed to generate and mix Sample i, and the processor calculates and records the concentration of the one or more additive chemical parameter(s) calculated for Sample i and/or the relative ratios of the two or more fluid samples that were mixed to produce Sample i, under Label i (step 805). The sensor then performs measurements on Sample i, and the results are recorded as Feature i (step 806). The current version of the machine learning system then makes a prediction (Prediction i) based on Feature i (step 807). The process is repeated (step 808) for all the N samples in the current batch. The processor then calculates an error estimate E (step 809) based on comparing Label i and Prediction i for all the N samples in the current batch. If the error estimate E is lower than the target error threshold E_{target}, E<E_{target} (step 812), then the current version of the machine learning system is saved (810), and the automatic training process stops (811), having successfully achieved the training goal. If the condition E<E_{target} is not satisfied at step 812, then the Features 1, . . . , N and the corresponding Labels 1, . . . , N from the current batch are added to the enlarged database (step 813), which is used to retrain the machine learning model (step 814). The process then returns to step 802, and is repeated until one of the exit conditions is met.

The choice of new sample combinations at step 804 may be made such as to provide a dense coverage of a portion of interest from the phase space of possible sample combinations. In a further related embodiment, the portion of interest from the phase space of possible sample combinations is determined based on the likelihood of the respective combinations being encountered in real-life situations.

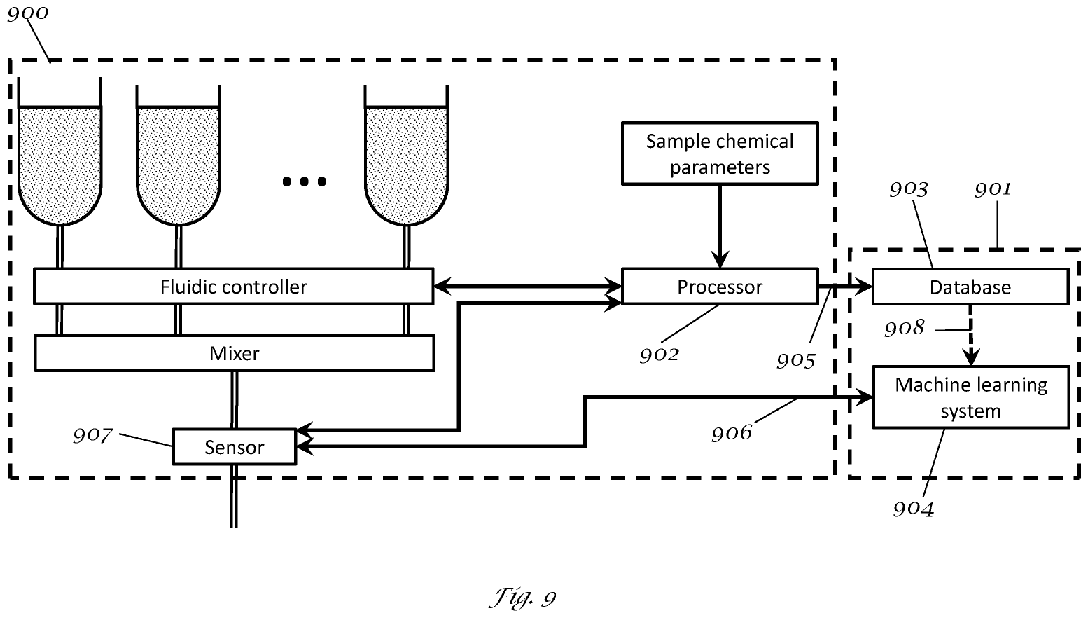

FIG. 9 displays an embodiment, wherein the database 903 and the machine learning system 904 are not physically part of the system 900, but are located on a remote server 901. The processor 902 is able to communicate 905 with the database 903 through a wired or wireless communication link, so as to transmit the result(s) of the one or more measurements performed by the first sensor 907, as feature(s), and the concentration of the one or more additive chemical parameter(s) of the homogenously mixed sample and/or the relative ratios of the two or more fluid samples, as label(s). Once data from a sufficient number of samples is acquired in the remote database 903, the remote machine learning system 904 is trained 908 on the data from the database. Sensor 907 can then communicate directly 906, via a wired or wireless communication link, with the remote learning system 904, to transmit measurement data and receive a predicted value of the one or more additive chemical parameter(s) of the homogenously mixed sample and/or the relative ratios of the two or more fluid samples.

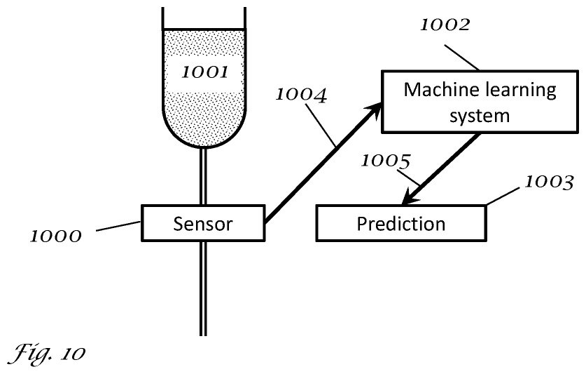

In another embodiment, schematically depicted in FIG. 10, the system includes a copy of the machine learning system 1002 trained on the data, configured for use with a second sensor 1000 that is similar to the first sensor, in type and resulting output given a sample (i.e., both sensors provide a measurement within a desired range of precision and accuracy). The second sensor accepts a sample 1001, which does not have a known concentration of the chemical parameters of interest, performs measurements on the sample 1001, transmits the measurements 1004 to the copy of the machine learning system, which uses the data to make predictions 1003 of the chemical parameters within the sample of interest 1001. These predictions may then be transmitted back to the sensor 1000, or other device, for display. The sample 1001 may be provided to the second sensor manually, or using an automated device such as, for example, an automatic dispenser, an autosampler, a pump, or a suction system.

The copy of the machine learning system 1002 may be located on a remote server and may be accessible via an application programming interface API or via a user interface, while the transmission of the measurements 1004 from the second sensor to the copy of the machine learning system, and of the predictions 1005 from the machine learning system back to the sensor, are performed over remote wired or wireless communication links.

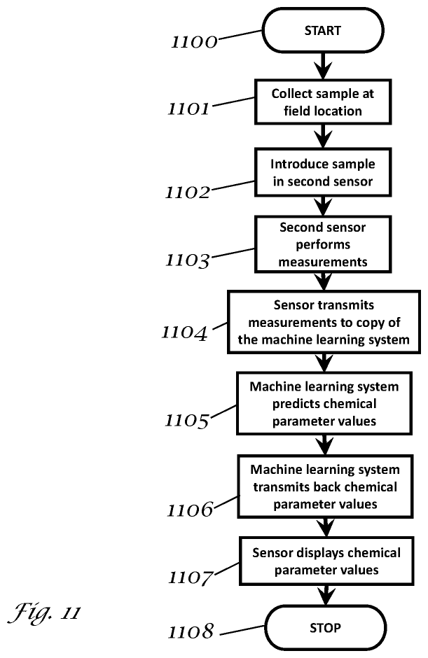

In various embodiments, the second sensor may be used in the field or in an operational environment. FIG. 11 depicts an exemplary protocol for using the second sensor in the field to analyze an unknown sample. Upon starting (step 1100), the operator collects a sample of interest (step 1101) at a field location and introduces it (step 1102) in the second sensor. The second sensor performs the measurements (step 1103) on the sample of interest. It then transmits (step 1104) the measurements to the copy of the machine learning system, which makes a prediction (step 1105) of the chemical parameter values of the sample of interest. The parameter predictions are the transmitted back (step 1106) to the sensor, which displays (step 1107) the results. This concludes (step 1108) the sample analysis protocol.

Embodiments can be implemented in whole or in part as a computer program product for use with a computer system. Such implementation may include a series of computer instructions fixed either on a tangible medium, such as a computer readable medium (e.g., a diskette, CD-ROM, ROM, USB disk, flash memory, magnetic disk or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., radio waves, microwaves, infrared light or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A system for generating machine learning training data sets for fluid monitoring applications, the system comprising:
two or more containers, each container for storing a fluid sample with a known concentration of one or more additive chemical parameters;
a fluidic controller for independently controlling the flow of the two or more fluid samples, through fluidic conduits, from their respective containers to a mixing unit, in such a way that the relative ratios of the two or more fluid samples delivered to the mixing unit is monitored;
a mixer for homogenizing the mixture of the two or more fluid samples in the mixing unit to create a homogenously mixed sample;
a first sensor for performing one or more measurements on the homogenously mixed sample;
a database; and
a processor configured to:
receive as an input, for each fluid sample, the concentration of the one or more additive chemical parameters;
instruct the fluidic controller and mixer so as to generate the homogenously mixed sample of the fluid samples;
determine a concentration of the one or more additive chemical parameters of the homogenously mixed sample from a combination of the relative ratios of the two or more fluid samples and of the known concentration of their respective additive chemical parameters;
obtain results of one or more measurements performed by the first sensor on the homogenously mixed sample; and
store in the database the result(s) of the one or more measurements performed by the first sensor, as feature(s), and the concentration of the one or more additive chemical parameter(s) of the homogenously mixed sample and/or the relative ratios of the two or more fluid samples, as label(s).

2. The system according to claim 1, wherein the fluidic controller is configured to provide fluid sample flow from each container to the mixing unit under the action of gravity, of an external pressure or force applied to the sample, and/or of a vacuum applied to the outlet.

3. The system according to claim 2, wherein the fluidic controller includes individual on-off valves for successively controlling the flow of each fluid sample from the containers to the mixing unit, and further includes one of a scale and a calibrated level meter for measuring the amount of each sample fluid that is being dispensed while the respective valve is in the on position.

4. The system according to claim 1, wherein the fluidic controller includes an individual metering pump and/or an automatic pipetting system, for controlling the individual amounts of the different fluid samples added to the mixing unit.

5. The system according to claim 1, wherein the fluidic controller is configured to control a flow rate of each individual fluid sample flowing towards the mixing unit.

6. The system according to claim 5, wherein the fluidic controller includes a controllable pump for controlling the flow rate of each fluid sample.

7. The system according to claim 5, wherein the fluidic controller includes one or multiple flow meters installed on the fluid conduits for measuring the flow rate of the fluid samples as they flow towards the mixing unit.

8. The system according to claim 5, wherein the fluidic controller is configured to have the ability to adjust the individual pressure or force applied to each fluid sample, so as to adjust its flow rate in the respective fluid conduit.

9. The system according to claim 8, wherein the fluidic controller is configured to apply individual pressures to each fluid sample through a pressurizing fluid in gas or liquid phase, said pressurizing fluid being separated from each fluid sample by one of a compliant partition, a piston, and a threaded bag or combinations thereof.

10. The system according to claim 8, wherein each fluidic conduit further includes a hydraulic resistor that is selected, based on the respective fluid sample viscosity and the maximum pressure and/or force that the controller can apply to the fluid sample, so as to limit the flow rate of the respective fluid sample to a desired maximum value.

11. The system according to claim 5, wherein the fluidic controller is configured to control the effective hydraulic resistance of each fluidic conduit, by the means of operating one of a regulating valve, modulating valve, variable constriction, pinch valve, ball valve, globe valve, and needle valve or combinations thereof.

12. The system according to claim 5, wherein the mixer is an in-line mixer configured to homogenize the different fluid sample streams on the fly, wherein the first sensor is an in-line sensor configured to measure the resulting homogenously mixed sample while it flows, and wherein the processor is configured to determine the relative ratios of the two or more fluid samples present in the homogenously mixed sample stream from their respective instantaneous flow rates.

13. The system according to claim 1, wherein at least one of the fluid conduits, mixing unit, mixer and first sensor is a microfluidic device with channels having lateral dimension between 1 μm and 1000 μm.

14. The system according to claim 1, wherein the processor automatically generates a selection of fluid sample combinations that are directed to a predetermined portion of the phase space of possible combinations.

15. The system according to claim 14, wherein the predetermined portion of the phase space targets ranges of samples combinations based on their likelihood of being encountered in real-life situations.

16. The system according to claim 1, wherein the database is located on a remote computer server which communicates with the processor via a wireless or wired communication link.

17. The system according to claim 1, further including a machine learning system trained on the data stored in the database, for predicting at least one of the one or more chemical parameters values and the relative ratios of two or more fluid samples, from measurements performed on new fluid samples.

18. The system according to claim 17, further including a copy of the machine learning system trained on the data, for remote use with a second sensor similar to the first sensor.

19. The system according to claim 17, wherein the fluid samples are acquired in an operational environment and do not have a known concentration of one or more additive chemical parameters.

20. The system according to claim 17, wherein the machine learning system is located on a remote computer server, the first sensor transmits the measurements to the server via a wireless or wired communication link, and the server transmits back the one or more chemical parameters values predicted by the machine learning system.

21. The system according to claim 17, wherein the machine learning system is at least partially based on an artificial neural network.

22. The system according to claim 17, where the machine learning system implements partial least squares regression.

23. The system according to claim 17, wherein the processor is further configured to determine an error estimate based on comparing the predicted and the determined parameter values for one or more new sample fluids with a known concentration of one or more additive chemical parameters, but that are not yet included in the database.

24. The system according to claim 23 wherein the processor is further configured to:
select new fluid sample combinations to generate new features and labels to include in an enlarged training database;
re-train the machine learning system on the enlarged training database;
recalculate a new error estimate, and
iteratively repeat the process of selecting, re-training and recalculating until the error estimate decreases below a desired precision threshold, or a maximum number of iterations is reached.

25. The system according to claim 1, wherein the two or more fluid samples are selected from one of:
surface water matrices likely to be encountered at a specific location and/or pollutant streams known to contaminate the respective surface water matrices,
hydrocarbon mixtures found in downhole oilfield exploration, refining operations and/or produced crude oils,
an alcoholic beverage combined with a product used to adulterate alcoholic beverages,
pure drinking water combined with pollutants, contaminants, chemical warfare agents, nerve agents, toxic and/or poisonous compounds, and
pure air combined with pollutants, chemical warfare agents, nerve agents, toxic and/or poisonous compounds.

26. The system according to claim 1, wherein the first sensor is one of:
an optical spectrometer, and the measurement includes an absorbance spectrum measured in the ultraviolet, visible, near infrared, mid infrared and/or far infrared spectral ranges,
a fluorescence spectrometer, and the measurement includes an emission spectrum, an excitation spectrum, or an excitation-emission matrix,
a Ramon spectrometer,
a chromatography system, and the measurement includes a chromatograph recorded with a detector, and
a chemical sensor array.

27. The system according to claim 1, further including a cleaning mechanism for cleaning and/or decontaminating the mixing unit, mixer and/or first sensor between successive measurements.

28. A method of generating machine learning training data sets for fluid monitoring applications, the method comprising:
mixing two or more fluid samples to generate a homogenously mixed sample such that the relative ratios of the two or more fluid samples that are mixed is known, each fluid sample having a known concentration of one or more additive chemical parameters;
determining a concentration of the one or more additive chemical parameters of the homogenously mixed sample from a combination of the relative ratios of the two or more fluid samples and of the known concentration of their respective additive chemical parameters;
obtaining results of one or more sensor measurements on the homogenously mixed sample; and
storing in a database the result(s) of the one or more measurements, as feature(s), and the concentration of the one or more additive chemical parameter(s) of the homogenously mixed sample and/or the relative ratios of the two or more fluid samples, as label(s).

29. The method according to claim 28, wherein each fluid sample is stored in a separate container, and wherein mixing two or more fluid samples includes providing flow of fluid sample from each container to a mixing unit under the action of gravity, of an external pressure or force applied to the sample, and/or of a vacuum or suction pump applied to the outlet.

30. The method according to claim 29, wherein providing flow of fluid sample from each container to the mixing unit includes controlling the flow of fluid sample with individual on-off valves, and further includes measuring the amount of each sample fluid that is being dispensed while the respective valve is in the on position.

31. The method according to claim 29, wherein providing fluid sample flow from each container to the mixing unit includes using an individual metering pump and/or an automatic pipetting system, for controlling the individual amounts of the different fluid samples added to the mixing unit.

32. The method according to claim 29, wherein providing fluid sample flow from each container to the mixing unit includes controlling a flow rate of each individual fluid sample flowing towards the mixing unit.

33. The method according to claim 32, wherein controlling the flow rate of each individual fluid sample flowing towards the mixing unit includes using a controllable pump for controlling the flow rate of each fluid sample.

34. The method according to claim 32, wherein controlling the flow rate of each individual fluid sample flowing towards the mixing unit includes measuring the flow rate of the fluid samples as they flow towards the mixing unit.

35. The method according to claim 32, wherein controlling the flow rate of each individual fluid sample flowing towards the mixing unit includes adjusting the individual pressure or force applied to each fluid sample.

36. The method according to claim 35, wherein adjusting the individual pressure or force applied to each fluid sample includes applying individual pressures to each fluid sample through a pressurizing fluid in gas or liquid phase, said pressurizing fluid being separated from each fluid sample by one of a compliant partition, a piston, and a threaded bag or combinations thereof.

37. The method according to claim 32, wherein controlling the flow rate of each individual fluid sample flowing towards the mixing unit includes using a hydraulic resistor.

38. The method according to claim 32, wherein controlling the flow rate of each individual fluid sample flowing towards the mixing unit includes controlling the effective hydraulic resistance using one of a regulating valve, a modulating valve, a variable constriction, a pinch valve, a ball valve, a globe valve, and a needle valve or combinations thereof.

39. The method according to claim 28, wherein mixing two or more fluid samples to generate a homogenously mixed sample includes homogenizing different fluid sample streams on the fly, wherein obtaining results of one or more sensor measurements on the homogenously mixed sample includes measuring the resulting homogenously mixed sample while it flows, and wherein the relative ratios of the two or more fluid samples present in the homogenously mixed sample stream is determined from their respective instantaneous flow rates.

40. The method according to claim 28, wherein the method includes using microfluidic device with channels having lateral dimension between 1 μm and 1000 μm.

41. The method according to claim 28, further comprising generating a selection of fluid sample combinations for mixing that are directed to a predetermined portion of the phase space of possible combinations.

42. The method according to claim 41, wherein the predetermined portion of the phase space targets ranges of samples combinations based on their likelihood of being encountered in real-life situations.

43. The method according to claim 28, wherein a processor controls and/or performs the mixing, determining, obtaining and storing, and the database is located on a remote computer server which communicates with the processor via a wireless or wired communication link.

44. The method according to claim 28, further comprising training a machine learning system on the data stored in the database.

45. The method according to claim 28, further comprising:
predicting, by the machine learning system, the one or more chemical parameters values and/or the relative ratios of two or more fluid samples, from measurements performed on new fluid samples not included in the training.

46. The method according to claim 45, further including a copy of the machine learning system trained on the data, for remote use.

47. The method according to claim 45, wherein the new fluid samples are acquired in an operational environment.

48. The method according to claim 44, wherein the machine learning system is located on a remote computer server, the method further comprising sending the results of the one or more sensor measurements to the server via a wireless or wired communication link, and transmitting back, by the server, the one or more chemical parameters values predicted by the machine learning system.

49. The method according to claim 44, wherein the machine learning system is at least partially based on an artificial neural network.

50. The method according to claim 44 where the machine learning system implements partial least squares regression.

51. The method according to claim 44, further comprising determining an error estimate based on comparing the predicted and the determined parameter values for new fluid sample combinations that are not yet included in the database, which have a known concentration of one or more additive chemical parameters.

52. The method according to claim 51, further comprising:
selecting new fluid sample combinations to generate new features and labels to include in an enlarged training database;
re-training the machine learning system on the enlarged training database;
recalculate a new error estimate, and
iteratively repeat the process of selecting, re-training and recalculating until the error estimate decreases below a desired precision threshold, or a maximum number of iterations is reached.

53. The method according to claim 28, wherein the two or more fluid samples are selected from one of:
surface water matrices likely to be encountered at a specific location and/or pollutant streams known to contaminate the respective surface water matrices,
hydrocarbon mixtures found in downhole oilfield exploration, refining operations and/or produced crude oils,
an alcoholic beverage combined with a product used to adulterate alcoholic beverages,
pure drinking water combined with pollutants, contaminants, chemical warfare agents, nerve agents, toxic and/or poisonous compounds, and
pure air combined with pollutants, chemical warfare agents, nerve agents, toxic and/or poisonous compounds.

54. The method according to claim 28, wherein obtaining results of one or more sensor measurements on the homogenously mixed sample includes using one of:
an optical spectrometer, and the measurement includes an absorbance spectrum measured in the ultraviolet, visible, near infrared, mid infrared and/or far infrared spectral ranges,
a fluorescence spectrometer, and the measurement includes an emission spectrum, an excitation spectrum, or an excitation-emission matrix,
a Raman spectrometer,
a chromatography system, and the measurement includes a chromatograph recorded with a detector, and
a chemical sensor array.

55. The method according to claim 28, wherein the method is performed by a system, the method further including cleaning and/or decontaminating the system between successive measurements.

56. The method of claim 28, performed using the system of claim 1.

57. A method of generating machine learning training data sets for fluid monitoring applications, performed using the system of claim 1.
